(19) United States
(12) Patent Application Publication
(10) Pub. No.: US 2022/0340451 A1
(43) Pub. Date: Oct. 27, 2022

(54) DEVICE FOR ULTRAVIOLET IRRADIATION OF FLUIDS WITH INTEGRATED CONTAINER

(71) Applicant: The University of British Columbia, Vancouver (CA)

(72) Inventor: Fariborz TAGHIPOUR, Burnaby (CA)

(21) Appl. No.: 17/747,986

(22) Filed: May 18, 2022

Related U.S. Application Data
(63) Continuation of application No. PCT/CA2020/051771, filed on Dec. 18, 2020.
(60) Provisional application No. 62/950,414, filed on Dec. 19, 2019.

(51) Int. Cl. C02F 1/32

(52) U.S. Cl. CPC C02F 1/325; C02F 2201/328; C02F 2201/3222; C02F 2303/04

(57) ABSTRACT

An apparatus for treating a fluid comprises a fluid container having a chamber for holding the fluid and an ultraviolet (UV) reactor. The UV reactor comprises: an inlet in fluid communication with the chamber; an outlet located above the inlet; a conduit extending longitudinally between the inlet and the outlet to facilitate fluid flow from the inlet to the outlet in a first direction having a component antiparallel to the direction of gravity; and a UV emitter optically oriented to emit UV radiation directed toward the fluid travelling through the conduit. At least part of the chamber is located above the outlet to cause the fluid to flow from the chamber to the inlet and through the conduit to the outlet due to force of gravity.

### REFERENCE TO RELATED APPLICATIONS

This application is a continuation of Patent Cooperation Treaty (PCT) application No. PCT/CA2020/051771 filed 18 Dec. 2020 which in turn claims priority from, and for the purposes of the United States of America the benefit under 35 U.S.C. § 119 of, U.S. Application No. 62/950,414 filed 19 Dec. 2019. All of the applications in this paragraph are hereby incorporated herein by reference for all purposes.

### TECHNICAL FIELD

The present invention relates to fluid treatment apparatus, and more particularly, to fluid treatment apparatus that include an ultraviolet (UV) reactor. Particular embodiments have example applications for treating and/or disinfecting water.

### BACKGROUND

It is common to use ultraviolet (UV) radiation for irradiating fluids flowing in UV reactors. In particular, it is common to use UV reactors for applications such as water and air purification.

Some UV reactor systems use low-pressure and/or medium-pressure mercury lamps to generate UV radiation. Systems like these are typically not energy efficient and not environmentally friendly. Other systems exist but may not be capable of providing adequate UV radiation dose or UV fluence (i.e. to the fluid being treated). Fluence or radiation dose is the fluence rate multiplied by the exposure time (i.e. the residence time of a fluid in the UV reactor while exposed to UV radiation). Fluence rate (in W/m²) is the radiant flux (power) passing from all directions through an infinitesimally small sphere of cross-sectional area dA, divided by dA.

Solid state radiation emitters, such as light emitting diodes (LEDs), are radiation sources that emit radiation of narrow bandwidth. LEDs may be designed to generate UV radiation at different wavelengths, which include wavelengths for DNA absorption as well as wavelengths that can be used for photocatalyst activation. Hence, UV-LED reactors are sometimes used to treat fluids at their point of use or point of entry with a flow-through reactor.

For water-use applications, it can be desirable to store the water before and/or after treatment. There remains a need for top-of-the-counter apparatus capable of storing the water prior to irradiation, providing adequate UV fluence rate to the water during irradiation, and storing the water after irradiation. There may be a desire for such apparatus to consume minimal or no power consumption for moving the water through the apparatus.

There is also a general desire, in the field of irradiation treatment of fluids, to enhance dose uniformity delivered to fluids flowing in or through a UV reactor.

There is also a general desire for apparatus that include both a UV reactor and other types of filters or methods for fluid treatment, particularly where water may be stored in the apparatus before and/or after treatment.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides an apparatus for treating a fluid, the apparatus comprising: a fluid container having a chamber for holding the fluid; and an ultraviolet (UV) reactor. The UV reactor comprises: an inlet in fluid communication with the chamber; an outlet located above the inlet; a conduit defined by a body, the conduit extending longitudinally between the inlet and the outlet to facilitate fluid flow from the inlet to the outlet in a first direction, the first direction having a component antiparallel to the direction of gravity; and a UV emitter for directing UV radiation toward the fluid travelling through the conduit. At least part of the chamber is located above the outlet to cause the fluid to flow from the chamber to the inlet and through the conduit to the outlet due to force of gravity.

The UV emitter may be optically oriented to emit UV radiation directed in a second direction, the second direction having a component parallel to the direction of gravity. The second direction may be antiparallel to the first direction. The first direction may be antiparallel to the direction of gravity and the second direction may be parallel to the direction of gravity.

The first and second directions may be the same direction. The first and second directions may be oriented antiparallel to the direction of gravity.

The chamber may be located entirely above the outlet of the UV reactor.

The apparatus may comprise a second fluid container having an inlet in fluid communication with the outlet of the UV reactor and a chamber for storing the treated fluid. The second fluid container may be shaped to define a cavity and the UV reactor may be removably inserted in the cavity to place the outlet of the UV reactor in fluid communication with the inlet of the second fluid container. The second fluid container may be shaped to receive and support the first fluid container on top of the second fluid container. The first fluid container may be removably mounted on top of the second fluid container to place the outlet of the first fluid container in fluid communication with the inlet of the UV reactor.

The UV emitter may comprise one or more solid-state radiation sources. The one or more solid-state radiation sources may be coupled to a thermally conductive substrate. The thermally conductive substrate may be in thermal contact with the fluid as the fluid flows from the inlet through the conduit to the outlet of the UV reactor.

The apparatus may further comprise one or more filters located in the fluid path between the first fluid container and the UV reactor. The one or more filters may comprise one or more materials filters selected from the group consisting of: a sediment filter, a carbon filter, and ion-exchange resin beads. The one or more filters may be annularly cylindrically shaped or located in an annular cylinder-shaped container and may be disposed around the UV reactor.

The apparatus may further comprise a flow distributor located at the outlet of the first container.

The apparatus may further comprise a control valve configured to regulate fluid flow through the UV reactor.

The apparatus may further comprise a sensor connected to the UV emitter to turn the UV emitter ON or OFF automatically. The sensor may be a flow sensor configured to turn the UV emitter ON when the flow sensor detects movement of fluid through the outlet of the chamber of the first container. The sensor may comprise a conductivity sensor having first and second conductive components that are electrically coupled when fluid flows between the first and the second conductive components. The sensor may be located near the bottom of the chamber of the first container to detect fluid flow near the bottom of the chamber and to turn the UV emitter ON upon detecting fluid flow near the bottom of the chamber. The apparatus may further comprise a hydrophobic material located near the sensors. The sensor may comprise a pressure sensor.

The UV reactor may comprise a rechargeable battery for powering the UV emitter. The rechargeable battery may be electrically connected to an external charging station through an electrically conductive medium of the second fluid container. The external charging station may comprise a hand-crank generator, a solar cell, an electricity generator, or an external battery.

Another aspect of the invention provides a fluid treatment apparatus comprising: a first fluid container having a first chamber; a second fluid container having a second chamber; and an ultraviolet (UV) reactor. The UV reactor comprises: an inlet in fluid communication with the first chamber; an outlet in fluid communication with the second chamber; a conduit defined by a body, the conduit extending longitudinally between the inlet and the outlet to facilitate fluid flow from the inlet to the outlet in a first direction; and a UV emitter for directing UV radiation into the conduit. The apparatus comprises a filter located in a fluid path between the first and second chambers. At least part of the first chamber is located above the outlet to facilitate fluid flow from the first chamber through the conduit and to the second chamber due to force of gravity.

The outlet may be located above the inlet. The first direction may have a component antiparallel to the direction of gravity.

The filter may comprise one or more materials filters selected from the group consisting of: a sediment filter, a carbon filter, and ion-exchange resin beads.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a schematic view of an ultraviolet (UV) reactor according to an example embodiment of the invention.

FIG. 2 is a side view of an exemplary embodiment of a fluid treatment apparatus comprising a UV reactor of the type shown in FIG. 1.

FIG. 3A is a perspective view of another exemplary embodiment of a fluid treatment apparatus comprising a UV reactor of the type shown in FIG. 1. FIG. 3B is a front view of the FIG. 3A dispenser.

FIG. 4A is a perspective view of another exemplary embodiment of a fluid treatment apparatus comprising a UV reactor of the type shown in FIG. 1. FIGS. 4B-E show various exemplary features of the FIG. 4A dispenser.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

One aspect of the invention relates to UV reactors that use UV light emitting diodes (LEDs) as the source of UV radiation to treat fluids flowing through the UV reactor. For the purposes of facilitating the description, the term “untreated fluid” may be used herein to refer to fluid that has not yet received UV radiation from UV emitters of the UV reactors described herein. Similarly, the term “treated fluid” may be used herein to refer to fluid that has received UV radiation from UV emitters of the UV reactors described herein. The terms “untreated fluid” and “treated fluid” should not be construed in the literal sense and are used in the description from time to time for brevity to distinguish between fluids flowing into a UV reactor and the same fluids flowing out of the UV reactor after they have been treated by the UV reactor. “Untreated fluid” may include fluids that have been treated previously by other filters or other fluid treatment systems.

In this application, unless the context dictates otherwise, the term antiparallel, when referring to two directions, should be understood to mean the two directions are parallel but oriented in opposing directions (i.e. two directions oriented at 180° from one another). Two directions that are parallel can have the same orientation or antiparallel orientations.

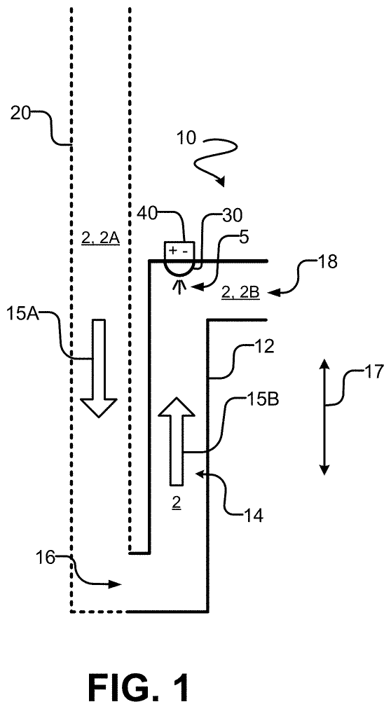

FIG. 1 is a schematic side cross-sectional view of an ultraviolet (UV) reactor 10 according to an example embodiment of the invention. UV reactor 10 has example applications for disinfecting fluid 2 (e.g. water) flowing through UV reactor 10. UV reactor 10 may be embodied as a part of a fluid treatment apparatus, a water dispenser or a water filter pitcher as described in more detail elsewhere herein.

As depicted in FIG. 1, UV reactor 10 comprises a body 12 defining a conduit 14 extending longitudinally (e.g. in a longitudinal direction 17) between an inlet 16 and an outlet 18. Fluid 2 (e.g. water) enters conduit 14 through inlet 16 and exits conduit 14 at outlet 18. UV reactor 10 comprises a UV emitter 30 optically oriented to direct UV radiation 5 toward fluid 2 as fluid 2 flows through conduit 14. Unless context dictates otherwise, the term “optically oriented” (as used herein) should be interpreted to imply that UV emitter 30 may include optical elements (e.g. lenses, reflectors, waveguides, etc.) located in the optical path between a UV radiation source and an output of UV emitter 30 to emit UV radiation 5 that is principally oriented in a particular direction (e.g. within 5° of solid angle from the particular direction). In some embodiments, radiation that is optically oriented in a particular direction may have a maximal intensity in that particular direction (e.g. within 5° of solid angle from the particular direction). In some embodiments, radiation that is optically oriented in a particular direction may have maximal irradiance in that particular direction (e.g. within 5° of solid angle from the particular direction). In some embodiments, radiation that is optically oriented in a particular direction may have maximal emitted power in that particular direction (e.g. within 5° of solid angle from the particular direction). In some embodiments, radiation that is optically oriented in a particular direction may be collimated in that particular direction (e.g. within 5° of solid angle from the particular direction). In the case of the illustrated embodiment, UV emitter 30 is optically oriented to direct UV radiation 5 toward fluid 2 as fluid 2 flows through conduit 14.

UV emitter 30 typically includes one or more solid-state radiation emitters such as UV-LEDs, although other types of UV radiation emitters are possible. Advantageously, UV-LEDs may be smaller, operated at lower power, less expensive to manufacture, and more environmentally friendly than other UV radiation sources (e.g. UV lamps). UV emitter 30 may include a waterproof casing that prevents fluid 2 from damaging the radiation sources of UV emitter 30.

In currently preferred embodiments, outlet 18 is located above (i.e., at a higher elevation than) inlet 16. In these embodiments, fluid 2 flows from inlet 16 to outlet 18 in a direction having a component antiparallel to the direction of the force of gravity. Inlet 16 may be connected to and may receive untreated fluid 2A from a pipe 20 that extends above outlet 18 as shown in FIG. 1. This relative location of pipe 20, inlet 16 and outlet 18 allows fluid 2 to flow down pipe 20, up through conduit 14, and out of outlet 18 due to force of gravity. That is, fluid 2 may flow from inlet 16 upwardly through conduit 14 and out of outlet 18 without the need for a pump or other active (i.e. energy-consuming) fluid moving devices.

UV emitter 30 is suitably located and suitably optically oriented to emit UV radiation 5 directed toward fluid 2 as fluid 2 flows from inlet 16 (in a direction having a component antiparallel to the direction of gravity) to outlet 18. That is, UV emitter 30 is optically oriented to emit UV radiation 5 directed toward fluid 2 as fluid 2 flows against the force of gravity through conduit 14. For example, fluid 2 may flow in an upward direction 15B against the force of gravity and UV emitter 30 may be optically oriented to emit UV radiation 5 directed in downward direction 15A as shown in FIG. 1. In other embodiments, UV emitter 30 may be located near inlet 16 (not shown) and optically oriented to emit UV radiation directed in upward direction 15B as fluid 2 flows from inlet 16 (in a direction having a component antiparallel to the direction of gravity—e.g. in upward direction 15B against the force of gravity) to outlet 18. Arrangements like these (where fluid is forced upwardly through reactor 10 (e.g. from inlet 16 upwardly through conduit 14 to outlet 18) may advantageously provide a more uniform residence time for fluid 2 flowing in UV reactor 10 and/or a more uniform UV fluence (i.e. dose) delivered to fluid 2 (when compared to forcing fluid through a reactor using a pump or some other active (energy-supplying) device or when allowing fluid flow through the reactor in the direction of gravity), thereby enhancing the performance of UV reactor 10.

In some embodiments, pipe 20 is integrally formed with and may be a part of conduit 14. In these embodiments, conduit 14 may be shaped to facilitate fluid flow (i.e. flow of fluid 2) in a first direction having a component parallel to the direction of force of gravity (i.e. a direction having a “downward” component) followed by a second direction having a component antiparallel to the direction of force of gravity (i.e. a direction having an “upward” component). For example, conduit 14 may be U-shaped to facilitate fluid flow in downward direction 15A followed by upward direction 15B.

Although not necessary, UV emitter 30 is typically located at a location that is more proximate to outlet 18 than inlet 16. In the example embodiment shown in FIG. 1, UV emitter 30 is located adjacent to outlet 18 and optically oriented to emit UV radiation 5 directed in downward direction 15A.

In some embodiments, UV emitter 30 is powered by a battery 40. Battery 40 may be physically located in close proximity to UV emitter 30 as shown in FIG. 1. Battery 40 may be a rechargeable battery. For example, battery 40 may be recharged using a hand-crank generator. As another example, battery 40 may be recharged using a solar cell (or a solar cell may be used to power UV emitter 30 directly). As another example, battery 40 may be recharged using a hydraulic power generator (or a hydraulic power generator may be used to power UV emitter 30 directly). UV reactor 10 may include a user interface (not shown) configured to inform the user of the status of battery 40.

Another aspect of the invention relates to fluid treatment apparatus that include UV reactor 10 or variations thereof. Advantageously, such apparatus may be operated without the need for a pump or other active (i.e. energy-consuming) fluid moving devices. Such apparatus may be embodied as, for example, household water dispensers, household water filter pitchers, portable water filters, etc.

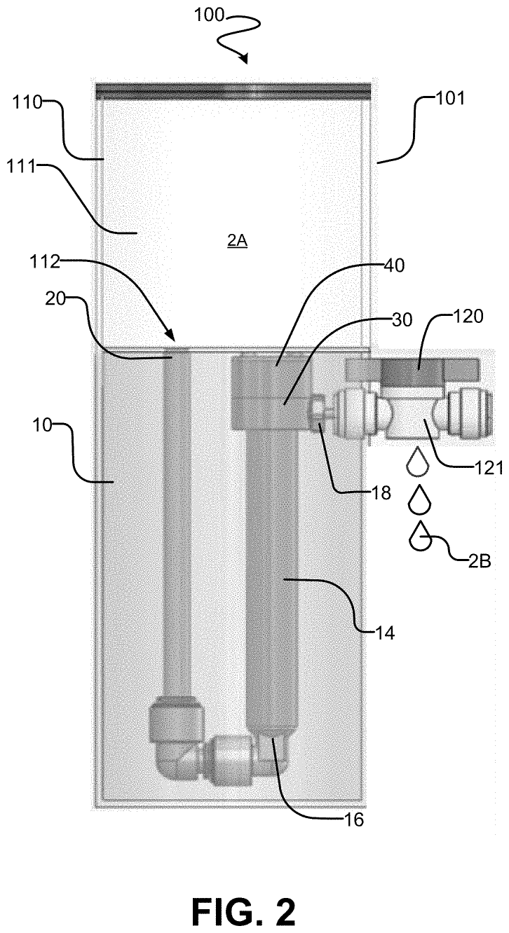

FIG. 2 is a side view of a dispenser 100 according to an example embodiment of the invention. Dispenser 100 comprises a fluid container 110 that holds (e.g. stores) untreated fluid 2A in a chamber 111 of fluid container 110, a UV reactor 10 that receives the untreated fluid 2A from fluid container 110 and then treats (e.g. disinfects) the unfiltered fluid 2A, and optionally a tap 120 that may be operated to dispense filtered fluid 2B (i.e. after it has been filtered by UV reactor 10).

As shown in FIG. 2, fluid container 110 comprises a container outlet 112 that is in fluid communication with inlet 16 of UV reactor 10. Unless context dictates otherwise, the term “in fluid communication” (as used herein) should be interpreted to mean a connection that allows fluid to flow from one component to another. For clarity, two elements that are “in fluid communication” do not need to be in direct physical contact with each other and may include additional elements (e.g. pipes, airgaps, etc.) located in the fluid path between the two elements. Two elements that are in “direct fluid communication” may be in “fluid communication” with one another and in physical contact with one another.

Fluid container 110 may be partially or entirely located above (i.e., at a higher elevation than) UV reactor 10. For example, fluid container 110 may be stacked or otherwise mounted directly on top of UV reactor 10 as shown in FIG. 2. Likewise, chamber 111 and container outlet 112 may be located above both inlet 16 and outlet 18 of UV reactor 10 as shown in FIG. 2. This allows untreated fluid 2A to flow out of chamber 111 (i.e. out of container outlet 112) into conduit 14 (i.e. through inlet 16) and then out of outlet 18 under the influence of force of gravity. Container outlet 112 may be in fluid communication with inlet 16 directly or through a short pipe, a control valve, etc.

In operation, untreated fluid 2A flows out of chamber 111 through container outlet 112 and enters conduit 14 through inlet 16. Untreated fluid 2A is treated (i.e. disinfected) by UV emitter 30 as it flows through conduit 14 in a direction having at least a component that is antiparallel to the direction of the force of gravity. Treated fluid 2B exits conduit 14 through outlet 18 (i.e. after fluid 2 receives UV radiation 5 from UV emitter 30). After exiting UV reactor 10, treated fluid 2B may be dispensed out of dispenser 100 through tap 120.

Tap 120 is in fluid communication with outlet 18 of UV reactor 10. In some embodiments, tap 120 comprises a valve 121 which may be operated to cause fluid 2 to flow therethrough. That is, tap 120 may comprise a valve 121 which may be moved between an open position that allows fluid 2 to flow therethrough and a closed position that prevents fluid 2 from flowing therethrough. When valve 121 is moved to its open position, fluid 2 may flow therethrough to thereby allow fluid 2 to flow through UV reactor 10.

UV reactor 10, fluid container 110 and/or tap 120 may be disposed inside of a housing 101 as shown in FIG. 2, although this is not necessary.

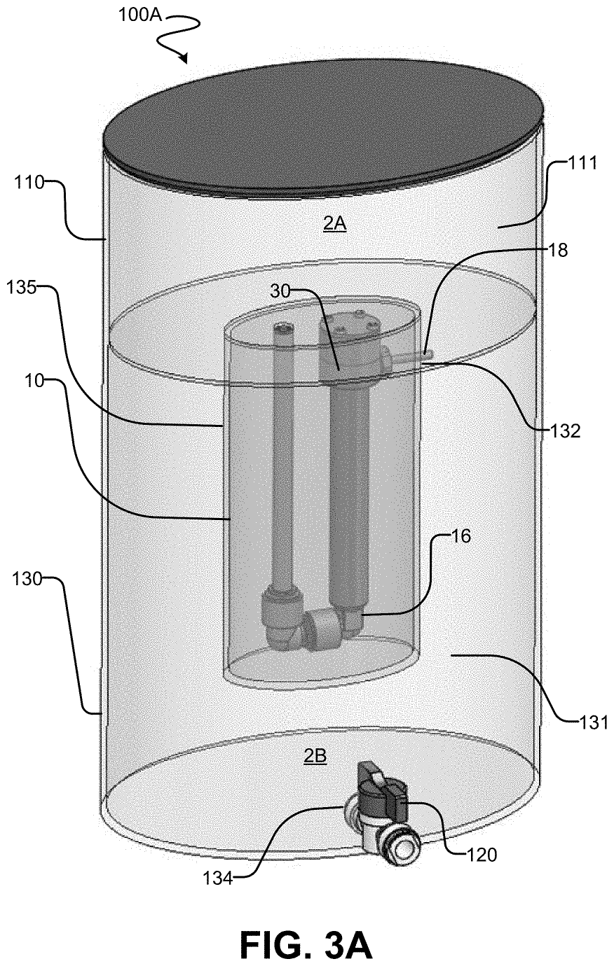

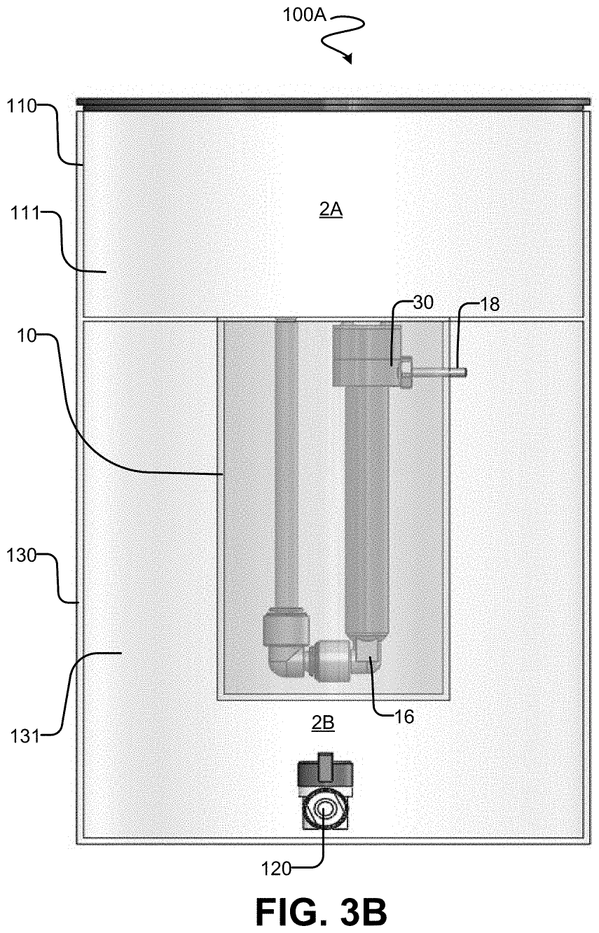

FIG. 3A is a perspective view of a dispenser 100A according to another example embodiment of the invention. FIG. 3B is a front view of the FIG. 3A dispenser 100A. Like dispenser 100, dispenser 100A comprises a fluid container 110 that holds untreated fluid 2A, a UV reactor 10 that receives the untreated fluid 2A from fluid container 110 and then treats (e.g. disinfects) the unfiltered fluid 2A, and optionally a tap 120 that may be operated to dispense filtered fluid 2B out of dispenser 100A.

Dispenser 100A comprises a second additional fluid container 130 that holds (e.g. stores) treated fluid 2B (after it has been filtered by UV reactor 10) in a chamber 131 of second fluid container 130. Second fluid container 130 includes a second fluid container inlet 132 in fluid communication with outlet 18 of UV reactor 10. Second fluid container 130 may also include a second fluid container outlet 134 in fluid communication with tap 120. Second fluid container inlet 132 may (but need not necessarily) be located at a higher elevation than second fluid container outlet 134 as shown in FIGS. 3A-3B. Second fluid container 130 stores treated fluid 2B in chamber 131 after it has been treated by UV reactor 10. Second fluid container 130 may be designed to store any volume of fluid 2B, including up to 1 L, 2 L, 3 L, 4 L, 5 L or any volume therebetween.

In some embodiments, second fluid container 130 is shaped to define a cavity 135 that may receive and physically support UV reactor 10. In these embodiments, UV reactor 10 may be removably insertable into cavity 135. That is, UV reactor 10 may be inserted into cavity 135 when dispenser 100A is in use and removed from cavity 135 when dispenser 100A is not in use. It may be desirable to remove UV reactor 10 from cavity 135 in some situations. For example, it may be desirable to remove UV reactor from cavity 135 to recharge UV reactor 10, to store UV reactor 10 at a suitable location, etc.

In some embodiments, inserting UV reactor 10 into cavity 135 places outlet 18 of UV reactor 10 in fluid communication with second fluid container inlet 132. Second fluid container inlet 132 may be located at a suitable location on second fluid container 130 to receive or otherwise be in fluid communication with outlet 18 of UV reactor 10 when UV reactor 10 is inserted in cavity 135.

In some embodiments, second fluid container 130 is shaped to support untreated fluid container 110. In these embodiments, untreated fluid container 110 may be mounted directly on top of second fluid container 130 (and on top of UV reactor 10 inserted in cavity 135) as shown in FIGS. 3A-B.

In some embodiments, mounting untreated fluid container 110 on top of second fluid container 130 places container outlet 112 in fluid communication with inlet 16 of UV reactor 10. Container outlet 112 may be located at a suitable location on untreated fluid container 110 to fluidly connect to inlet 16 when untreated fluid container 110 is mounted on top of second fluid container 130 (and on top of UV reactor 10 inserted in cavity 135). Mounting untreated fluid container 110 on top of second fluid container 130 in this manner can help prevent UV reactor 10 from sliding out of cavity 135 when dispenser 100A is in use. This advantageously provides a compact and modular design for dispenser 100.

UV reactor 10, untreated fluid container 110 and/or second fluid container 130 may be disposed inside of a housing, although this is not necessary.

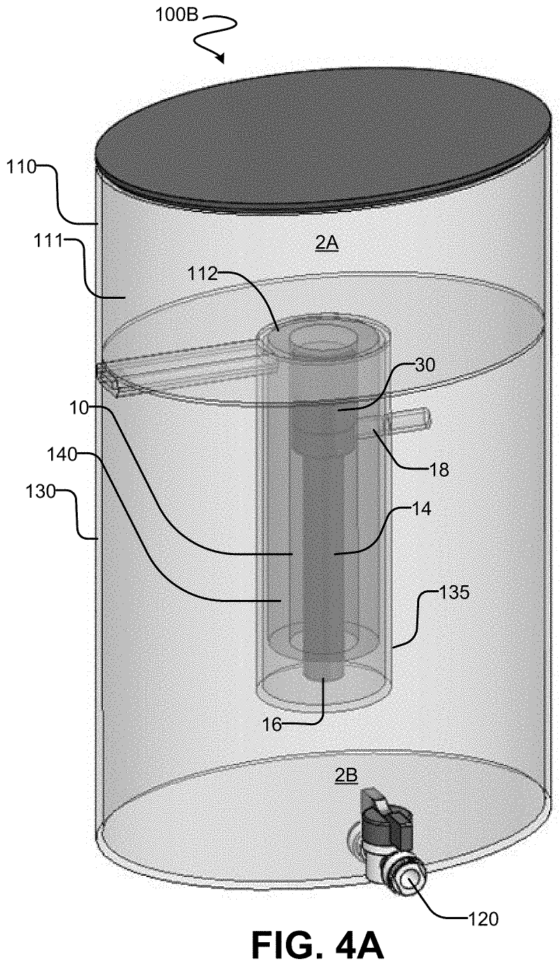

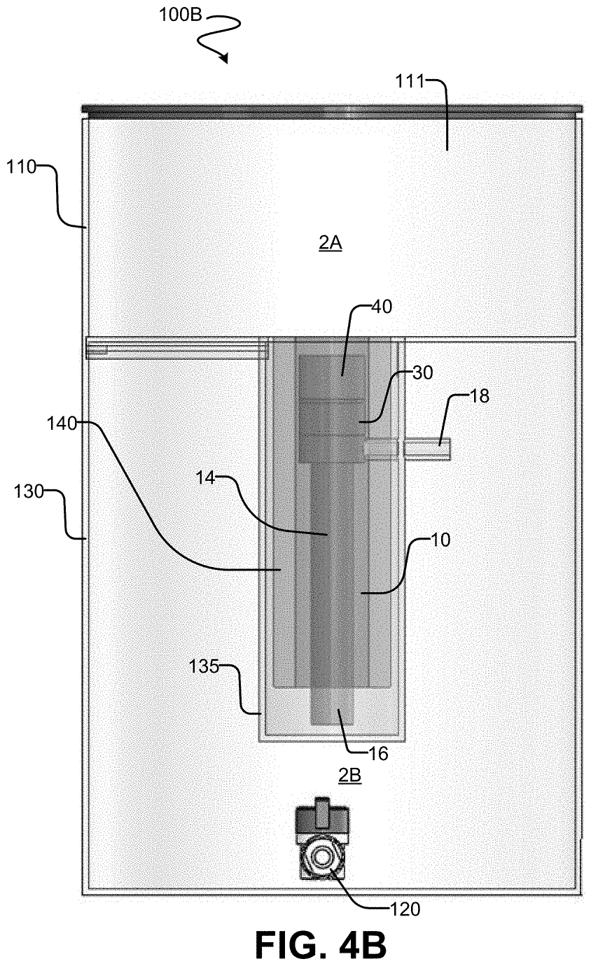

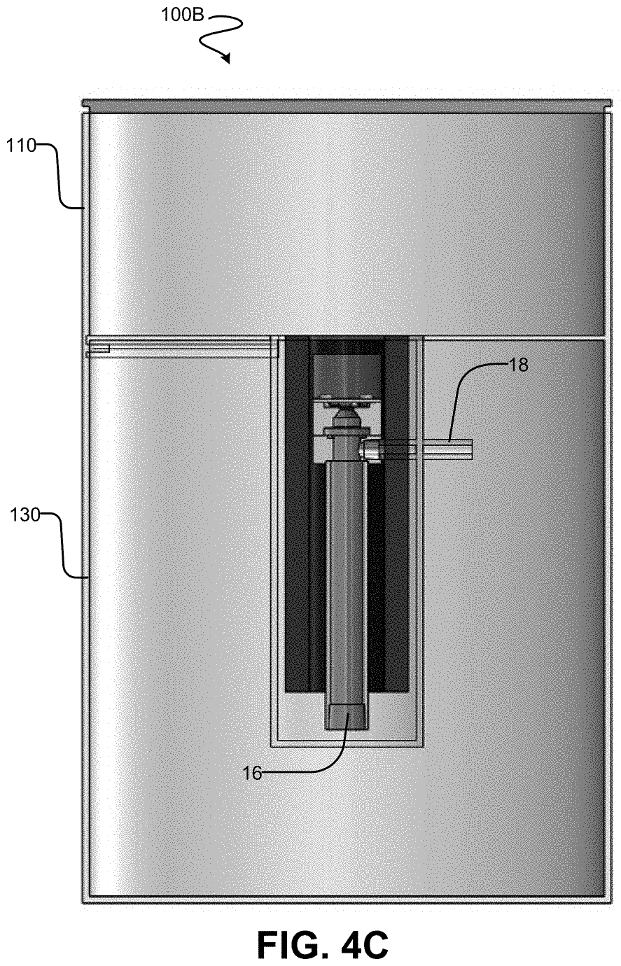

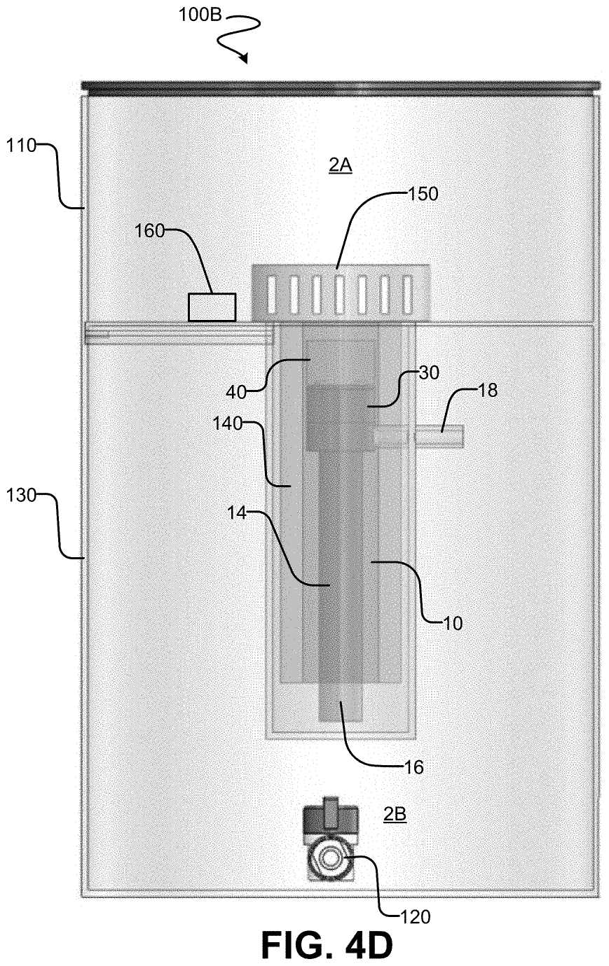

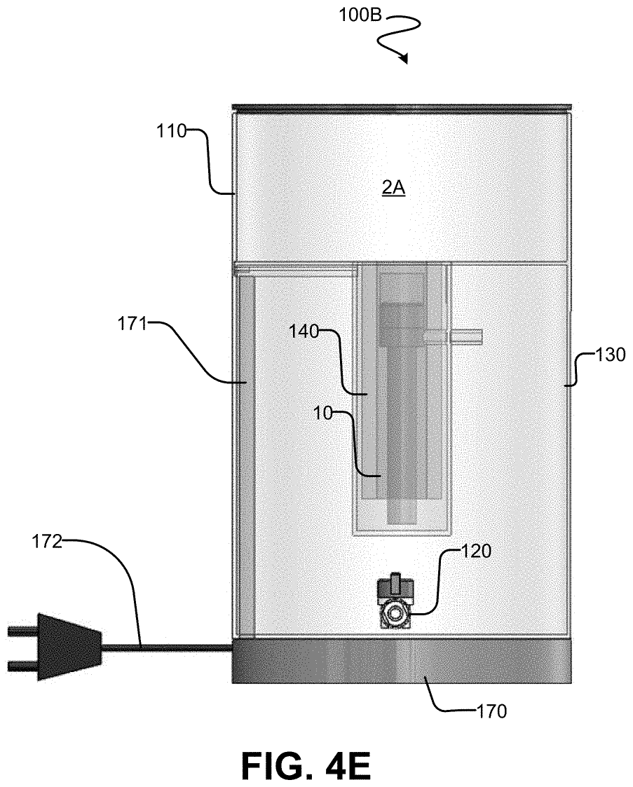

FIG. 4A is a perspective view of a dispenser 100B according to another example embodiment of the invention. FIGS. 4B-C are front views of the FIG. 4A dispenser 100B. Like dispenser 100A, dispenser 100B comprises a first fluid container 110 (for storing untreated fluid 2A) mounted on top of a second fluid container 130 (for storing treated fluid 2B), a UV reactor 10 located in a cavity 135 of the second fluid container 130, and optionally a tap 120 that may be operated to dispense filtered fluid 2B out of dispenser 100B.

Dispenser 100B comprises a one or more filters 140 located in the fluid path between outlet 112 of the first fluid container 110 and inlet 16 of UV reactor 10. Filters 140 may include one or a combination of sediment filters, activated charcoal filters, carbon filters, ion-exchange resin beads, composite filters, etc. Each of filters 140 may include one or more filter media. For example, filters 140 may include a first filter having a first filter media (e.g. an activated carbon filter) placed in series with a second filter having a second filter media (e.g. an ion exchange resin bed). As another example, an individual filter 140 may include both a first filter media and a second filter media. Filters 140 may individually or collectively remove particles as small as 5 microns or smaller from fluid 2A.

Advantageously, filters 140 may remove chemical contaminants such as organic and inorganic contaminants from untreated fluid 2A before untreated fluid 2A flows through UV reactor 10. For example, filters 140 may comprise carbon-based filters that remove taste and odor causing chemicals from fluid 2A. As another example, filters 140 may comprise cation exchange materials (e.g. water softeners) that remove unwanted minerals (e.g. positively charged ions such as calcium and magnesium) from fluid 2A and, optionally, replace the unwanted minerals with other minerals (e.g. sodium). As another example, filters 140 may comprise anion exchange materials that remove unwanted anions (e.g. negatively charged ions such as arsenic and nitrate) and replace them with other anions (e.g. chloride).

Filters 140 may extend around UV reactor 10 to form an annular cylinder around UV reactor 10. Filters 140 may be located in an annular cylinder-shaped container provided around UV reactor 10, as shown in FIGS. 4A-E. In the example embodiments shown in FIGS. 4A-E, fluid 2A flows in a direction having a component parallel to the direction of the force of gravity (e.g. vertically downwards) through filters 140 before fluid 2A enters UV reactor 10 through inlet 16. Filters 140 may be wrapped snuggly around UV reactor 10. Alternatively, filters 140 may be wrapped loosely around UV reactor 10 to provide some spacing between materials filters 140 and UV reactor 10.

Dispenser 1006 and other dispensers or fluid treatment apparatus described herein may optionally include a flow distributor 150 that helps distribute the flow of fluid 2A from untreated fluid container 110 to filters 140 and/or UV reactor 10. Flow distributor 150 may be configured to distribute the flow of fluid 2A evenly across filters 140 and/or control the rate of fluid flow through UV reactor 10. Flow distributor 150 may be located at the outlet 112 of fluid container 110 as shown in FIG. 4D, although this is not necessary. Dispenser 1006 and other dispensers or fluid treatment apparatus described herein may include other devices that control the flow of fluid 2 through UV reactor 10. These devices include, but are not limited to, control valves, flow controllers, and pressure controllers.

Dispenser 100B and other dispensers or fluid treatment apparatus described herein may also optionally include sensor(s) configured to automatically turn UV emitter 30 ON and OFF. For example, dispenser 100B may comprise a conductivity sensor 160 located at or near the bottom of chamber 111 of container 110 as shown in FIG. 4D. Conductivity sensor 160 may comprise two spaced apart conductive components (e.g. two pieces of metal) that form an electrical connection when fluid 2 is present therebetween. Conductivity sensor 160 may be connected to UV emitter 30 to turn UV emitter 30 ON when conductivity sensor 160 detects a fluid in chamber 111 and to turn UV emitter 30 OFF when conductivity sensor 160 detects a lack of fluid in chamber 111. Dispenser 100B may comprise hydrophobic materials located near conductivity sensor 160 to remove any residual fluids that may cause an unwanted connection (between the two conductive components of conductivity sensor 160). Conductivity sensor 160 may be connected to UV emitter 30 through a controller (not shown). The controller may be configured to delay turning UV emitter 30 off (upon conductivity sensor 160 a lack of fluid in chamber 111). The controller may be configured to automatically re-fill fluid container 110 when the fluid level in chamber 111 is below a threshold level (which may be detected, for example, by a level controller).

Dispenser 100B and other dispensers or fluid treatment apparatus described herein may include other types of sensors such as flow sensors connected to turn UV emitter 30 ON when fluid 2 flows through UV reactor 10.

Dispenser 100B and other dispensers or fluid treatment apparatus described herein may include a removable charging station 170 electrically connected to UV emitter 30 through an electrically conductive medium 171 (e.g. a wire) as shown in FIG. 4E. Charging station 170 may include a plug 172 that can be electrically connected to an outlet or USB charger to either recharge the battery 40 of UV reactor 10 or deliver electric power directly to UV emitter 30.

Embodiments of this disclosure are directed to embodiments of fluid treatment apparatus, fluid dispensers and/or UV reactors that provide enhanced dose uniformity by controlling both fluidic and optical environments. Some embodiments have been described above with reference to particular types of radiation sources, particular fluids and/or particular types of radiation. For example, the radiation source may have been described above as a solid-state radiation source such as UV-LED, fluids may have been described above as water, and the radiation may have been described above as UV radiation. Unless claimed, these examples are provided for convenience and not intended to limit the present disclosure. Accordingly, any structural embodiments described in this disclosure may be utilized with any analogous radiation sources, fluids, and/or radiation types.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. An apparatus for treating a fluid, the apparatus comprising:
a fluid container having a chamber for holding the fluid; and
an ultraviolet (UV) reactor comprising:
an inlet in fluid communication with the chamber;
an outlet located above the inlet;
a conduit defined by a body, the conduit extending longitudinally between the inlet and the outlet to facilitate fluid flow from the inlet to the outlet in a first direction, the first direction having a component antiparallel to the direction of gravity; and
a UV emitter for directing UV radiation toward the fluid travelling through the conduit, wherein the UV emitter is optically oriented to emit UV radiation directed in a second direction, the second direction substantially parallel with the longitudinal extension of the conduit,
wherein at least part of the chamber is located above the outlet to cause the fluid to flow from the chamber to the inlet and through the conduit to the outlet due to force of gravity.

2. The apparatus of claim 1, wherein the second direction substantially parallel with the longitudinal extension of the conduit is parallel to the direction of gravity.

3. The apparatus of claim 2, wherein the second direction is antiparallel to the first direction.

4. The apparatus of claim 3, wherein the first direction is antiparallel to the direction of gravity and wherein the second direction is parallel to the direction of gravity.

5. The apparatus of claim 1, wherein the first and second directions are the same direction.

6. The apparatus of claim 5 wherein the first and second directions are oriented antiparallel to the direction of gravity.

7. The apparatus of claim 1, wherein the chamber is located entirely above the outlet of the UV reactor.

8. The apparatus of claim 1, further comprising a second fluid container having an inlet in fluid communication with the outlet of the UV reactor and a chamber for storing the treated fluid.

9. The apparatus of claim 8, wherein the second fluid container is shaped to define a cavity and wherein the UV reactor is removably inserted in the cavity to place the outlet of the UV reactor in fluid communication with the inlet of the second fluid container.

10. The apparatus of claim 9, wherein the second fluid container is shaped to receive and support the first fluid container on top of the second fluid container.

11. The apparatus of claim 10, wherein the first fluid container is removably mounted on top of the second fluid container to place the outlet of the first fluid container in fluid communication with the inlet of the UV reactor.

12. The apparatus of claim 1, wherein the UV emitter comprises one or more solid-state radiation sources.

13. The apparatus of claim 12, wherein the one or more solid-state radiation sources is coupled to a thermally conductive substrate, and wherein the thermally conductive substrate is in thermal contact with the fluid as the fluid flows from the inlet through the conduit to the outlet of the UV reactor.

14. The apparatus of claim 1, further comprising one or more filters located in the fluid path between the first fluid container and the UV reactor.

15. The apparatus of claim 14, wherein the one or more filters comprise one or more materials filters selected from the group consisting of: a sediment filter, a carbon filter, and ion-exchange resin beads.

16. The apparatus of claim 14, wherein the one or more filters are annularly cylindrically shaped or located in an annular cylinder-shaped container and are disposed around the UV reactor.

17. The apparatus of claim 1, further comprising a flow distributor located at the outlet of the first container.

18. The apparatus of claim 1, further comprising a control valve configured to regulate fluid flow through the UV reactor.

19. The apparatus of claim 1, further comprising a sensor connected to the UV emitter to turn the UV emitter ON or OFF automatically.

20. The apparatus of claim 19, wherein the sensor is a flow sensor configured to turn the UV emitter ON when the flow sensor detects movement of fluid through the outlet of the chamber of the first container.

21. The apparatus of claim 19, wherein the sensor is a conductivity sensor having first and second conductive components that are electrically coupled when fluid flows between the first and the second conductive components.

22. The apparatus of claim 19, wherein the sensor is located near the bottom of the chamber of the first container to detect fluid flow near the bottom of the chamber and to turn the UV emitter ON upon detecting fluid flow near the bottom of the chamber.

23. The apparatus of claim 19, further comprising a hydrophobic material located near the sensors.

24. The apparatus of claim 19, wherein the sensor is a pressure sensor.

25. The apparatus of claim 1, wherein the UV reactor comprises a rechargeable battery for powering the UV emitter.

26. The apparatus of claim 25, wherein the rechargeable battery is electrically connected to an external charging station through an electrically conductive medium of the second fluid container.

27. The apparatus of claim 26, wherein the external charging station comprises a hand-crank generator, a solar cell, an electricity generator, or an external battery.

28. A fluid treatment apparatus comprising:
a first fluid container having a first chamber;
a second fluid container having a second chamber;
an ultraviolet (UV) reactor comprising:
an inlet in fluid communication with the first chamber;
an outlet in fluid communication with the second chamber;
a conduit defined by a body, the conduit extending longitudinally between the inlet and the outlet to facilitate fluid flow from the inlet to the outlet in a first direction; and
a UV emitter for directing UV radiation into the conduit wherein the UV emitter is optically oriented to emit UV radiation directed in a second direction, the second direction substantially parallel with the longitudinal extension of the conduit; and
a filter located in a fluid path between the first and second chambers;
wherein at least part of the first chamber is located above the outlet to facilitate fluid flow from the first chamber through the conduit and to the second chamber due to force of gravity.

29. The apparatus of claim 28 wherein the outlet is located above the inlet.

30. The apparatus of claim 28 wherein the first direction has a component antiparallel to the direction of gravity.

31. The apparatus of claim 28 wherein the filter comprises one or more materials filters selected from the group consisting of: a sediment filter, a carbon filter, and ion-exchange resin beads.

32. The apparatus of claim 28 herein wherein the UV reactor, the filter and the first and second fluid containers are supported in a single integrated housing.

33. The apparatus of claim 32 wherein the single integrated housing comprises a handle or is moveable by a human user using a single hand.
